(19) United States
(12) Patent Application Publication
(10) Pub. No.: US 2003/0162783 A1
(43) Pub. Date: Aug. 28, 2003

(54) Method and composition for the treatment of pain

(76) Inventors: Vernon Alford, Lawrenceville, NJ (US); Thomas Michael Bare, West Chester, PA (US); Dean Gordon Brown, Wilmington, DE (US); Frances Marie McLaren, Wilmington, DE (US); Megan Murphy, Wilmington, DE (US); Rebecca Ann Urbanek, Wilmington, DE (US); Wenhua Xiao, Montreal (CA)

Correspondence Address: ASTRA ZENECA PHARMACEUTICALS LP, GLOBAL INTELLECTUAL PROPERTY, 1800 CONCORD PIKE, WILMINGTON, DE, 19850-5437, US

(21) Appl. No.: 10/168,745

(22) PCT Filed: Dec. 19, 2000
(86) PCT No.: PCT/SE00/02605

(30) Foreign Application Priority Data
Dec. 23, 1999 (US) 60171906
Sep. 29, 2000 (US) 60236835

(51) Int. Cl. A61K 31/503

(52) U.S. Cl. 514/248

(57) ABSTRACT

A method for the treatment of pain is disclosed comprising administration of a pain-ameliorating effective amount of any compound according to structural diagram I;

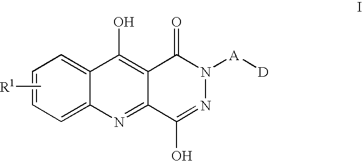

wherein: A, D and R¹ are as defined in the specification. Also disclosed are pharmaceutical compositions comprising a pain-ameliorating effective amount of a compound in accord with structural diagram I.

### FIELD OF THE INVENTION

This invention relates to the treatment or prevention of pain or nociception.

### RELATED ART

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. This range of sensations, as well as the variation in perception of pain by different individuals, renders a precise definition of pain difficult, however, many individuals suffer with severe and continuous pain.

Pain that is caused by damage to neural structures is often manifest as a neural supersensitivity or hyperalgesia and is termed “neuropathic” pain. Pain can also be “caused” by the stimulation of nociceptive receptors and transmitted over intact neural pathways, such pain is termed “nociceptive” pain.

The level of stimulation at which pain becomes noted is referred to as the “pain threshold.” Analgesics are pharmaceutical agents which relieve pain by raising the pain threshold without a loss of consciousness. After administration of an analgesic drug a stimulus of greater intensity or longer duration is required before pain is experienced. In an individual suffering from hyperalgesia an analgesic drug may have an anti-hyperalgesic effect. In contrast to analgesics, agents such as local anaesthetics block transmission in peripheral nerve fibers thereby blocking awareness of pain. General anaesthetics, on the other hand, reduce the awareness of pain by producing a loss of consciousness.

Tachykinin antagonists have been reported to induce antinociception in animals, which is believed to be analogous to analgesia in man (Maggi et al, J. Auton. Pharmacol. (1993) 13, 23-93). In particular, non-peptide NK-1 receptor antagonists have been shown to produce such analgesia. For example, the NK-1 receptor antagonist RP 67,580 produced analgesia with potency comparable to that of morphine (Garret et al, Proc. Natl. Acad. Sci. USA (1993) 88, 10208-10212).

The opioid analgesics are a well-established class of analgesic agents with morphine-like actions. Synthetic and semi-synthetic opioid analgesics are derivatives of five chemical classes of compound: phenanthrenes; phenylheptylamines; phenylpiperidines; morphinans; and benzomorphans. Pharmacologically these compounds have diverse activities, thus some are strong agonists at the opioid receptors (e.g. morphine); others are moderate to mild agonists (e.g. codeine); still others exhibit mixed agonist-antagonist activity (e.g. nalbuphine); and yet others are partial agonists (e.g. nalorphine). Whilst an opioid partial agonist such as nalorphine, (the N-alkyl analogue of morphine) will antagonize the analgesic effects of morphine, when given alone it can be a potent analgesic in its own right.

Of all of the opioid analgesics, morphine remains the most widely used, but, in addition to its therapeutic properties, it has a number of drawbacks including respiratory depression, decreased gastrointestinal motility (resulting in constipation), nausea and vomiting. Tolerance and physical dependence also limit the clinical uses of opioid compounds.

Aspirin and other salicylate compounds are frequently used in treatment to interrupt amplification of the inflammatory process in rheumatoid diseases and arthritis and temporarily relieve the pain. Other drug compounds used for these purposes include phenylpropionic acid derivatives such as Ibuprofen and Naproxen, Sulindac, phenyl butazone, corticosteroids, antimalarials such as chloroquine and hydroxychloroquine sulfate, and fenemates (J. Hosp. Pharm., 36:622 (May 1979)). These compounds, however, are ineffective for neuropathic pain.

Available therapies for pain also have drawbacks. Some therapeutic agents require prolonged use before an effect is experienced by the patient. Other existing drugs have serious side effects in certain patients, and subjects must be carefully monitored to ensure that any side effects are not unduly threatening. Most existing drugs provide only temporary relief from pain and must be taken consistently on a daily or weekly basis. With disease progression the amount of medication needed to alleviate the pain often increases, thus increasing the potential for adverse side effects.

NMDA receptors are defined by the binding of N-methyl-D-aspartate (NMDA) comprise a receptor/ion channel complex with several different identified binding domains. NMDA itself is a molecule structurally similar to glutamate (Glu) which binds at the glutamate binding suite and is highly selective and potent in activating the NMDA receptor (Watkins (1987); Olney (1989)).

Many compounds are known that bind at the NMDA/Glu binding site (for example CPP, DCPP-ene, CGP 40116, CGP 37849, CGS 19755, NPC 12626, NPC 17742, D-AP5, D-AP7, CGP 39551, CGP-43487, MDL-100,452, LY-274614, LY-233536, and LY233053). Other compounds, referred to as non-competitive NMDA antagonists, bind at other sites in the NMDA receptor complex (examples are phencyclidine, dizocilpine, ketamine, tiletamine, CNS 1102, dextromethorphan, memantine, kynurenic acid, CNQX, DNQX, 6,7-DCQX, 6,7-DCHQC, R(+)-HA-966, 7-chloro-kynurenic acid, 5,7-DCKA, 5-iodo-7-chloro-kynurenic acid, MDL-28,469, MDL-100,748, MDL-29,951, L-689,560, L687,414, ACPC, ACPCM, ACPCE, arcaine, diethylenetriamine, 1,10-diaminodecane, 1,12-diaminododecane, ifenprodil, and SL-82.0715). These compounds have been extensively reviewed by Rogawski (1992) and Massieu et. al., (1993), and articles cited therein.

In addition to its physiological function, glutamate (Glu) can be neurotoxic. Glu neurotoxicity is referred to as “excitotoxicity” because the neurotoxic action of Glu, like its beneficial actions, is mediated by an excitatory process (Olney (1990); Choi (1992)). Normally, when Glu is released at a synaptic receptor, it binds only transiently and is then rapidly removed from the receptor by a process that transports it back into the cell. Under certain abnormal conditions, including stroke, epilepsy and CNS trauma, Glu uptake fails and Glu accumulates at the receptor resulting in a persistent excitation of electrochemical activity that leads to the death of neurons that have Glu receptors. Many neurons in the CNS have Glu receptors, so excitotoxicity can cause an enormous amount of CNS damage.

Acute excitotoxicity injury can occur as a result of ischemic events, hypoxic events, trauma to the brain or spinal cord, certain types of food poisoning which involve an excitotoxic poison such as domoic acid, and seizure-mediated neuronal degeneration, which can result from persistent epileptic seizure activity (status epilepticus). A large body of evidence has implicated the NMDA receptor as one receptor subtype through which Glu mediates a substantial amount of CNS injury, and it is well established that NMDA antagonists are effective in protecting CNS neurons against excitotoxic degeneration in these acute CNS injury syndromes (Choi (1988); Olney (1990)).

In addition to neuronal damage caused by acute insults, excessive activation of Glu receptors may also contribute to more gradual neurodegenerative processes leading to cell death in various chronic neurodegenerative diseases, including Alzheimer's disease, amyotrophic lateral sclerosis, AIDS dementia, Parkinson's disease and Huntington's disease (Olney (1990)). It is generally considered that NMDA antagonists may prove useful in the therapeutic management of such chronic diseases.

In the 1980's it was discovered that PCP (also known as “angel dust”) acts at a “PCP recognition site” within the ion channel of the NMDA Glu receptor. PCP acts as a non-competitive antagonist that blocks the flow of ions through the NMDA ion channel. More recently it has become evident that drugs which act at the PCP site as non-competitive NMDA antagonists are likely to have psychotomimetic side effects. Further, it is now recognized that certain competitive and non-competitive NMDA antagonists can cause similar pathomorphological effects in rat brain (Olney et. al., (1991); Hargreaves et. al., (1993)). Such compounds also have psychotomimetic effects in humans (Kristensen et. al., (1992); Herrling (1994); Grotta (1994)).

The glycine binding site of the NMDA receptor complex is distinguishable from the Glu and PCP binding sites. Also, it has recently been discovered that NMDA receptors occur as several subtypes which are characterized by differential properties of the glycine binding site of the receptor. Many compounds that bind at the NOVA receptor glycine site, useful for the treatment of stroke and neurodegenerative conditions, have been described in U.S. Pat. Nos. 5,604,227; 5,733,910; 5,599,814; 5,593,133; 5,744,471; 5,837,705 and 6,103,721.

### SUMMARY OF THE INVENTION

It has now been discovered that certain compounds which exhibit the property of binding to the NMDA receptor glycine site have utility for the amelioration of pain and particularly for the amelioration of neuropathic pain.

Therefore, the invention provides a method for the treatment of pain comprising administering a pain-ameliorating effective amount of any compound according to structural diagram I;

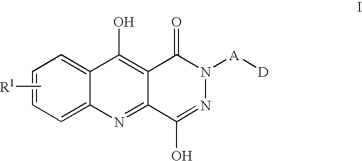

wherein: A is (CH₂)ₙ where n has a value selected from 0, 1, 2, 3 or 4; D is selected from a 5- or 6-membered heteroaryl moiety or a benz-derivative thereof, having 1, 2 or 3 ring atoms selected from oxygen, nitrogen or sulfur, and R¹ is halo.

In particular embodiments of the invention the method comprises administering pain-ameliorating effective amounts of a compound according to structural diagram I wherein: D is selected from pyridyl, quinolyly, pyrazinyl, pyradizinyl, furanyl, benz[b]furanyl, imidazolyl, oxazolyl, thienyl, benz[b]thienyl and thiazolyl.

In more particular embodiments of the invention the method comprises administering a pain-ameliorating effective amount of a compound according to structural diagram II wherein:

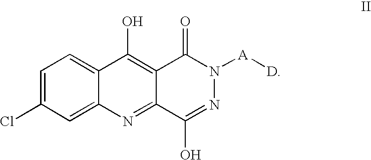

Still more particular embodiments of the invention are those where the method comprises treatment with a compound in accord with structural diagram 11 and D is selected from pyridyl, quinolyly, pyrazinyl, pyradizinyl, furanyl, benz[b]furanyl, imidazolyl, oxazolyl, thienyl, benz[b]thienyl and thiazolyl.

Yet more particular embodiments of the invention are those where the method comprises treatment with an exemplary compound specifically disclosed herein.

Yet other aspects of the invention are pharmaceutical compositions which contain a compound in accord with structural diagram I; the use of compounds in accord with structural diagram I for the preparation of medicaments and pharmaceutical compositions, and a method comprising binding a compound of the invention to the NMDA receptor glycine site of a warm-blooded animal, such as a human being, so as to beneficially inhibit the activity of the NMDA receptor.

### DETAILED DESCRIPTION OF THE INVENTION

Compounds of the invention are those within the scope of the generic description and particularly those compounds exemplified hereafter.

Suitable pharmaceutically-acceptable salts of compounds of the invention include acid addition salts such as methanesulphonate, fumarate, hydrochloride, hydrobromide, citrate, tris(hydroxymethyl)aminomethane, maleate and salts formed with phosphoric and sulphuric acid. In other embodiments, suitable salts are base salts such as an alkali metal salts for example sodium, alkaline earth metal salts for example calcium or magnesium, organic amine salts for example triethylamine, morpholine, N-methylpiperidine, N-ethylpiperidine, procaine, dibenzylamine, choline, N,N-dibenzylethylamine or amino acids such as lysine.

Another aspect of the invention is a process for making compounds of the invention, which process comprises the following steps:

a) Preparing a Boc-protected hydrazine according to one of the procedures shown in the following scheme:

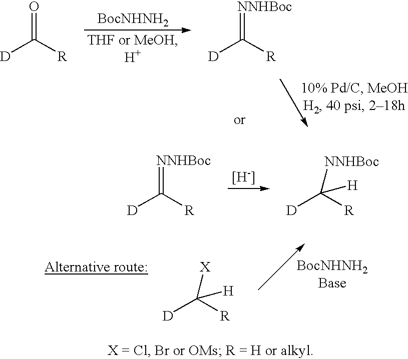

b) coupling said Boc-protected hydrazine and cyclizing the product according to the process of the following scheme to form a compound according to structural diagram I:

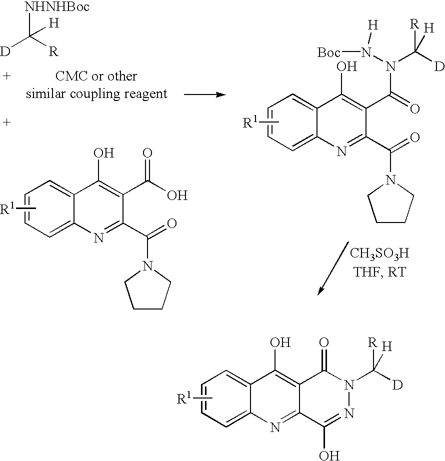

wherein:

CMC is 1-cyclohexyl-3-(2-morpholinoethyl)carbodiimide metho-p-toluenesulfonate;

the “R/H/D” group is the “-A-D” moiety of structural diagram I;

and throughout the foregoing process:

R¹ is as defined for structural diagram I.

To use a compound of the invention or a pharmaceutically-acceptable salt thereof for the therapeutic treatment, which may include prophylactic treatment, of pain in mammals, which may be humans, the compound can be formulated in accordance with standard pharmaceutical practice as a pharmaceutical composition.

Suitable pharmaceutical compositions that contain a compound of the invention may be administered in conventional ways, for example by oral, topical, parenteral, buccal, nasal, vaginal or rectal administration or by inhalation. For these purposes a compound of the invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions. A preferred route of administration is orally by tablet or capsule.

In addition to a compound of the present invention a pharmaceutical composition of this invention may also contain one or more other pharmacologically-active agents, or such pharmaceutical composition may be simultaneously or sequentially co-administered with one or more other pharmacologically-active agents.

Pharmaceutical compositions of this invention will normally be administered so that a pain-ameliorating effective daily dose is received by the subject. The daily dose may be given in divided doses as necessary, the precise amount of the compound received and the route of administration depending on the weight, age and sex of the patient being treated and on the particular disease condition being treated according to principles known in the art. A preferred dosage regime is once daily.

A further embodiment of the invention provides a pharmaceutical composition which contains a compound of the structural diagram I as defined herein or a pharmaceutically-acceptable salt thereof, in association with a pharmaceutically-acceptable additive such as an excipient or carrier.

A yet further embodiment of the invention provide the use of a compound of the structural diagram I, or a pharmaceutically-acceptable salt thereof, in the manufacture of a medicament useful for binding to the NMDA receptor glycine site in a warm-blooded animal such as a human being.

Still another embodiment of the invention provides a method of binding a compound of the invention to the NMDA receptor glycine site of a warm-blooded animal, such as a human being, in need of treatment for pain, which method comprises administering to said animal an effective amount of a compound of structural diagram I or a pharmaceutically-acceptable salt thereof.

Definitions:

When used herein the term “alkyl” includes both straight and branched chain alkyl groups but references to individual alkyl groups such as “propyl” refer to the straight chain moiety.

When used herein the term “halo” means fluoro, chloro, bromo and iodo.

When used herein the term “aryl” means an unsaturated carbon ring or a benz-derivative thereof. Particularly, aryl means phenyl, naphthyl or biphenyl. More particularly aryl means phenyl.

When used herein the term “heteroaryl” or “heteroaryl ring” means, unless otherwise further specified, a monocyclic-, bicyclic- or tricyclic- 5-14 membered ring that is unsaturated or partially unsaturated, with up to five ring heteroatoms selected from nitrogen, oxygen and sulphur wherein a —CH₂— group can optionally be replaced by a —C(O)—, and a ring nitrogen atom may be optionally oxidized to form the N-oxide. Examples of such heteroaryls include thienyl, furyl, pyranyl, pyrrolyl, imidazolyl, pyrazolyl, thiazolyl, oxazolyl, isoxazolyl, pyridyl, pyridyl-N-oxide, oxopyridyl, oxoquinolyl, pyrimidinyl, pyrazinyl, oxopyrazinyl, pyridazinyl, indolinyl, benzofuranyl, benzimidazolyl, benzothiazolyl, quinolyl, isoquinolinyl, quinazolinyl, xanthenyl, quinoxalinyl, indazolyl, benzofuranyl and cinnolinolyl.

When used herein the term “heterocyclyl” or “heterocyclic ring” means, unless otherwise further specified, a mono- or bicyclic- 5-14 membered ring, that is totally saturated, with up to five ring heteroatoms selected from nitrogen, oxygen and sulphur wherein a —CH₂— group can optionally be replaced by a —C(O)—. Examples of such heterocyclyls include morpholinyl, pyrrolidinyl, imidazolidinyl, pyrazolidinyl, piperidinyl, piperazinyl, homopiperidinyl, homopiperazinyl and quinuclidinyl.

When used herein, where optional substituents are selected from “one or more” groups it is to be understood that this encompasses compounds where all substituents are chosen from one of the specified groups and compounds where substituents are chosen from more than one of the specified groups.

Generally in the methods, processes and examples described herein:

concentrations were carried out by rotary evaporation in vacuo;

operations were carried out at ambient temperature, that is in the range 18-26° C. and under a nitrogen atmosphere;

column chromatography (by the flash procedure) was performed on Merck Kieselgel silica (Art. 9385) unless otherwise stated;

yields are given for illustration only and are not necessarily the maximum attainable;

the structure of the end-products of the formula I were generally confirmed by NMR and mass spectral techniques, proton magnetic resonance spectra were determined in DMSO-d₆ unless otherwise stated using a Varian Gemini 2000 spectrometer operating at a field strength of 300 MHz; chemical shifts are reported in parts per million downfield from tetramethylsilane as an internal standard (δ scale) and peak multiplicities are shown thus: s, singlet; bs, broad singlet; d, doublet; AB or dd, doublet of doublets; t, triplet, dt, double of triplets, m, multiplet; bm, broad multiplet; fast-atom bombardment (FAB) mass spectral data were obtained using a Platform spectrometer (supplied by Micromass) run in electrospray and, where appropriate, either positive ion data or negative ion data were collected, in this application, (M+H)⁺ is quoted; IR data was obtained with a Nicolet Avatar 360 FT-IR;

intermediates were not generally fully characterized and purity was in general assessed mass spectral (MS) or NMR analysis.

The following abbreviations and definitions when used, have the meanings, as follows:

| | |
|---|---|
| CDCl₃ | is deuturated chloroform; |
| CMC | is 1-cyclohexyl-3-(2-morpholinoethyl)carboiimide |
| | metho-p-toluenesulfonate; |
| DCM | is dichloromethane; |
| DCU | is dicyclohexyl urea; |
| DHC | is 1,3-dicyclohexylcarboiimide; |
| DMAP | is 4-(dimethylamino)pyridine; |
| DMF | is N,N-dimethylformamide; |
| DMSO | is dimethylsulphoxide; |
| m/s | is mass spectroscopy; |
| NMP | is N-methylpyrrolidinone; |
| NMR | is nuclear magnetic resonance; |
| p.o. | is per os; |
| THF | is tetrahydrofuran, and |
| t.i.d. | is three times daily. |

The examples and tests described herein are intended to illustrate but not limit the invention.

### EXAMPLES

### Example 1

### 7-Chloro-4-hydroxy-2-(4-pyridylmethyl)-1,2,5,10-tetrahydropyridazino[4,5-b]quinoline-1,10-dione methanesulfonate

(tert-Butoxy)-N-[(4-pyridylmethyl)amino]carboxamide

To a stirred solution of tert-butylcarbazate (174 g, 1.36 mole) and dry DMF (400 mL) under nitrogen was added triethylamine (108 mL, 0.78 mole) followed by 4-picolyl chloride hydrochloride (40.0 g, 0.243 mole). The reaction mixture was then heated at 75° C. for 5 hours and allowed to cool to room temperature. The reaction mixture was diluted with water (2 L) and the resulting mixture extracted with ethyl acetate (4×500 mL). The combined ethyl acetate extracts were concentrated under reduced pressure and the residue was dissolved in diethyl ether (1 L). The resulting solution was washed successively with water (3×400 mL) and brine (400 mL) and then dried over Na₂SO₄. The Na₂SO₄ was filtered off and the filtrate was concentrated under reduced pressure to give an amber oil (130.4 g). This product was purified by flash chromatography on silica gel eluting with hexane: ethyl acetate (1:1) to give the title compound as a solid off-white foam (24.46 g, 45%). ¹H NMR (300 MHz, DMSO-d₆): δ 1.36 (s, 9H); 3.90 (d, 2H, J=4.0 Hz); 5.04 (d, 1H, J=4.0 Hz); 7.34 (d, 1H, J=4.5 Hz); 8.48 (d, 1H, J=4.5 Hz).

Dimethyl 7-chloro4-hydroxyquinoline-2,3-dicarboxylate:

A stirred mixture of methyl 2-amino-4-chlorobenzoate (2.50 g, 13.5 mmol) and dimethyl acetylenedicarboxylate (2.05 g, 14.4 mmol) in tert-butanol (22 ml) was refluxed for 7 hours under a nitrogen atmosphere. After adding additional dimethyl acetylenedicarboxylate (1.16 g, 8.13 mmol) and refluxing another 2.5 hours, the reaction mixture was allowed to cool to room temperature and potassium tert-butoxide (1.56 g, 13.9 mmol) was added in one portion. A precipitate formed and the resulting mixture was refluxed for 1.5 hours. The mixture was cooled to room temperature and filtered to separate the solids, which were washed with tert-butanol and diethyl ether. The solids were dissolved in water and acidified with 1 N sulfuric acid to form a precipitate. The resulting mixture was extracted with DCM and the combined extracts were washed with brine and water, dried over MgSO₄, filtered and concentrated to give a green solid. Recrystallization of this material from methanol provided the title compound (1.15 g, 47%) as an off-white solid, mp 232-233° C.; MS (Cl):296.(M+H). Analysis for C₁₃H₁₀ClNO₅: Calc'd: C, 52.81; H, 3.41; N, 4.74; Found: C, 52.75; H, 3.47; N, 4.69.

3-Carbomethoxy-7-chloro4-hydroxyquinoline-2-carboxylic Acid:

To a stirred suspension of dimethyl 7-chloro-4-hydroxyquinoline-2,3-dicarboxylate (1.0 g, 3.38 mmol) in water (20 mL) was added an aqueous solution of sodium hydroxide (0.27 g, 6.75 mmol). Upon addition, the suspension dissolved. The reaction mixture was warmed to 60° C. for 1 hour. After this time the reaction was cooled to room temperature and acidified with concentrated hydrochloric acid. The product was then extracted into diethyl ether and ethyl acetate. The organic extracts were dried over MgSO₄, filtered and concentrated in vacuo to provide the title compound as a solid (900 mg). This material was purified by recrystallization employing an ethyl acetate/hexane co-solvent system to provide the title compound (571 mg, 60%) as a white solid mp 296° C. (dec); MS (CI)=238 (M+H). Analysis for C₁₂H₈NO₅Cl.0.45CH₃CO₂CH₂CH₃.0.10H₂O: Calc'd: C, 51.30; H, 3.68; N 4.34, Found: C, 51.28; H, 3.62; N 3.97 ¹ H NMR 8.22 (d, J=8.7 Hz, 1H), 7.92 (d, J=1.8 Hz, 1H), 7.28 (dd, J=8.7, 1.8 Hz, 1H), 3.90 (s, 3H).

3-Carbomethoxy-2-pyrrolidinocarbamide-7-chloro-4-hydroxyquinoline:

To a suspension of 3-carbomethoxy-7-chloro-4-hydroxyquinoline-2-carboxylic acid (2.25 g, 8.0 mmol) in THF (20 mL) at ambient temperature under a N₂ atmosphere was added DHC (1.65 g, 8.0 mmol) and pyrrolidine (0.596 g, 8.4 mmol). The reaction was stirred room temperature for 15 hours after which time the by-product urea was removed via filtration. The desired product was purified via flash column chromatography employing 5% methanol in chloroform to provide the title compound (2.52 g, 94.3%) as a tan solid, mp=215° C.; MS (CI): 335 (M+H). 300 MHz ¹H NMR (DMSO-d₆): δ 8.12 (d, J=8.7 Hz, 1H), 7.60 (d, 1H, J=1.8 Hz), 7.47 (dd, 1H, J=8.8, 2.0 Hz), 3.69 (s, 3H), 3.40-3.49 (m, 2H), 3.27-3.33 (m, 2H), 1.80-1.96 (m, 4H).

7-Chloro-4-oxo-2-(pyrrolidinylcarbonyl)hydroquinoline-3-carboxylic Acid:

To a suspension of 3-carbomethoxy-2-pyrrolidinocarbamide-7-chloro-4-hydroxy quinoline (2.52 g, 7.5 mmol) in de-ionized water (40 mL) was added dropwise a solution (20 mL) of an aqueous potassium hydroxide (882 mg, 15.75 mmol). Upon complete addition, the reaction was warmed to 60° C. After 3 hours, the reaction was filtered to remove a small amount of insoluble material. The filtrate was then acidified to pH=1 which yield a white precipitate. The solid was isolated by vacuum filtration, washed with water, and dried at 30° C. in vacuo for 16 hours. This provided the title compound (1.5 g, 64%) as a white solid, mp=225-8° C.; MS (CI): 321 (M+H). 300 MHz ¹H NMR (DMSO-d₆): δ 8.28 (d, J=8.8 Hz, 1H), 7.77 (s, 1H), 7.64 (d, 1H, J=8.7), 3.52-3.57 (m, 2H), 3.17-3.19 (m, 2H), 1.83-1.98 (m, 4H).

N-[(tert-butoxy)carbonylamino][7-chloro-4-oxo-2-(pyrrolidinylcarbonyl)(3-hydroquinolyl)]-N-(4-pyridylmethyl)carboxamide.

To a stirred mixture of 7-chloro-4-oxo-2-(pyrrolidinylcarbonyl)hydroquinoline-3-carboxylic acid (24.29 g, 75.73 mmol) and dry THF (1175 mL) under nitrogen was added CMC (50.55 g, 119.34 mmol) in portions (35 g followed by 15.55 g after 10 min.). After stirring the reaction mixture for an additional 20 minutes, a solution of (tert-butoxy)-N-[(4-pyridylmethyl)amino]carboxamide (22.0 g, 98.5 mmol) and THF (580 mL) was rapidly added and the mixture was stirred overnight. The reaction mixture was filtered and the filter cake was washed with DCM (300 mL). The filtrate and washings were combined and additional DCM (800 mL) was added. The resulting solution was washed with water (2×500 mL) and then dried over Na₂SO₄, filtered and concentrated under reduced pressure to give 28.90 g of yellow foam. This foam was treated with diethyl ether (800 mL) and the resulting mixture was stirred and then filtered. The filter cake was dried at 45° C. in vacuo to give the desired compound (24.3 g, 61%) as a yellow powder.

7-Chloro-4-hydroxy-2-(4-pyridylmethyl)-1,2,5,10-tetrahydropyridazino[4,5-b]quinoline-1,10-dione Methanesulfonate.

To a stirred mixture of N-[(tert-butoxy)carbonylamino][7-chloro-4-oxo-2-(pyrrolidinylcarbonyl)(3-hydroquinolyl)]-N-(4-pyridylmethyl)carboxamide (24.0 g, 45.62 mmol) and dry THF (960 mL) under nitrogen was added methanesulfonic acid (120 mL, 177.7 g, 1.85 moles) all at once. The mixture was stirred overnight and then filtered to separate the solids. The collected solids were successively washed with THF (2×100 mL), methanol (2×50 mL), and diethyl ether (100 mL). The filter cake (13.4 g) was then suspended in methanol (250 mL) and the resulting mixture sonicated for 20 minutes and then filtered. The collected solids were washed with methanol (2×100 mL) and diethyl ether (100 mL) and then dried at 45° C. in vacuo to give the title compound (12.1 g, 59%) as a yellow powder, m.p. >250° C. ¹H NMR (300 MHz, DMSO-d₆): δ 2.32 (s, 3H), 5.36 (s, 2H); 7.49 (dd, 1H, J=8.1 Hz, J=2.1 Hz); 7.86 (d, 1H, J=6.6 Hz); 8.06 (d, 1H, J=2.1 Hz); 8.12 (d, 1H, J=8.1 Hz); 8.82 (d, 1H, J=6.6 Hz); 12.6 (br s, 1H); 12.84 (br s, 1H). Calc'd. for C₁₇H₁₁ClN₄O₃.CH₃SO₃H.0.8H₂O: C, 46.47; H, 3.60; N, 12.04 Found: C, 46.39; H, 3.65; N, 11.98.

### Example 2

### 7-Chloro-4-hydroxy-2-(3-pyridylmethyl)-1,2,5,10-tetrahydropyridazino[4,5-b]quinoline-1,10-dione Methanesulfonate

(tert-Butoxy)-N-[3-pyridylmethyl)amino]carboxamide.

To a stirred solution of tert-butylcarbazate (203.6 g, 1.54 mole) and dry DMF (300 mL) under nitrogen was added triethylamine (128 mL, 0.92 mole) followed by 3-picolyl chloride hydrochloride (50.0 g, 0.30 mole) as a slurry in DMF (300 mL). The reaction mixture was heated at 75° C. for 3 hours, cooled to room temperature, and diluted with water (2.4 L). The resulting mixture was extracted with diethyl ether (3×800 mL). The aqueous layer was saturated with salt and extracted with diethyl ether (3×800 mL). The combined extracts were washed with water (1×1L), brine (1×1L) and then dried over Na₂SO₄. The Na₂SO₄ was filtered off and the filtrate was concentrated under reduced pressure. The product was purified by flash chromatography on silica gel eluting with diethyl ether to give the title compound as an off-white solid (23.3 g, 34%). ¹H NMR (300 MHz, DMSO-d₆): δ 1.36 (s, 9H); 3.88 (d, 2H, J=4.0 Hz); 4.96 (d, 1H, J=4.0 Hz); 7.33 (dd, 1H, J=7.7 Hz, J=4.8 Hz); 7.71 (d, 1H, J=7.7 Hz); 7.44 (d, 1H, J=4.7 Hz); 8.49 (s, 1H).

N-[(tert-butoxy)carbonylamino][7-chloro-4-oxo-2-(pyrrolidinylcarbonyl)(3-hydroquinolyl)]-N-(3-pyridylmethyl)carboxamide

To a stirred mixture of 7-chloro-4-oxo-2-(pyrrolidinylcarbonyl)hydroquinoline-3-carboxylic acid, Example 1, (20 g, 62.4 mmol) and THF (800 mL) under nitrogen was added CMC (40.0 g, 94.4 mmol). A solution of (tert-butoxy)-N-[(3-pyridylmethyl)amino]carboxamide (20.9 g, 93.6 mmol) and THF (450 mL) was rapidly added and the mixture stirred overnight. The reaction mixture was filtered and the filter cake was washed with THF. The filter cake was slurried with DCM and filtered. The filtrates were combined and evaporated under reduced pressure. The residue was dissolved in DCM, dried over Na₂SO₄, filtered and evaporated under reduced pressure to give a foam. The foam was stirred with diethyl ether (200 mL) and filtered. The filter cake was sonicated with diethyl ether (200 mL), filtered, washed with diethyl ether and was dried at 40° C. in vacuo to give the title compound as an off-white powder (32.8 g, 100%).

7-Chloro-4-hydroxy-2-(3-pyridylmethyl)-1,2,5,10-tetrahydropyridazino[4,5-b]quinoline-1,10-dione Methanesulfonate.

To a stirred solution of N-[(tert-butoxy)carbonylamino][7-chloro-4-oxo-2-(pyrrolidinylcarbonyl)(3-hydroquinolyl)]-N-(3-pyridylmethyl)carboxamide (32.8 g, mole) and THF (1 L) under nitrogen was added methanesulfonic acid (150 mL, 222 g, 2.31 moles) over 10 minutes. The mixture was stirred overnight and then filtered to separate the solids. The collected solids were washed with THF. The filter cake was suspended in methanol, sonicated (30 minutes) and filtered. The solids were resuspended in methanol, sonicated (30 minutes) and filtered. The collected solids were washed with methanol and then dried at 100° C. in vacuo to give the title compound (19.4 g, 66%) as a white solid, m.p. >300° C. ¹H NMR (300 MHz, DMSO-d₆): δ 2.33 (s, 3H); 5.29 (s, 2H)); 7.46 (dd, 1H, J=9.0 Hz, J=2.1 Hz); 7.94 (dd, 1H, J=9.0 Hz, J=5.6 Hz); 8.04 (d, 1H, J=1.8 Hz); 8.16 (d, 1H J=8.7 Hz); 8.37 (d, 1H, J=8.1 Hz); 8.82 (d, 1H J=4.8 Hz); 8.89 (s, 1H). Calc'd. for C₁₇H₁₁ClN₄O₃.CH₃SO₃H.H₂O: C, 46.11; H, 3.66; N, 11.95 Found: C, 46.34; H, 3.61; N, 11.94

### Example 3

### 7-Chloro-4-hydroxy-2-(2-pyridylmethyl)-1,2,5,10-tetrahydropyridazino[4,5-b]quinoline-1,10-dione Methanesulfonate

(tert-Butoxy)-N-[(2-pyridylmethyl)amino]carboxamide.

To a stirred solution of tert-butylcarbazate (174 g, 1.53 mole) and dry DMF (400 mL) under nitrogen was added triethylamine (130 mL, 0.94 mole) followed by 2-picolyl chloride hydrochloride (54.0 g, 0.33 mole). The reaction mixture was allowed to stir at ambient temperature for 1 hour, then heated at 70° C. for 3 hours and allowed to cool to room temperature. The reaction mixture was diluted with 1:1 mixture of ethyl acetate/diethyl ether and washed with brine and extracted. The aqueous layer was monitored by TLC (eluant: 100% diethyl ether) and was extracted several times with ethyl acetate (200 mL) until no product was observed. The combined organic extracts were washed with brine, dried over Na₂SO₄ and filtered. The filtrate was concentrated under reduced pressure to give an amber oil (˜100 g) which crystallized. The material was triturated with 1:1 diethyl ether/hexanes, filtered and dried under reduced pressure to afford the title compound as an off-white solid (33.4 g, 45% yield). ¹H NMR (300 MHz, DMSO-d⁶): δ 1.38 (s, 9H); 3.96 (d, 2H, J=4.0 Hz); 4.98 (d, 1H, J=4.0 Hz); 7.24 (dd, 1H, J=7.8 Hz, J=7.8 Hz); 7.48 (d, 1H); 7.74 (dd, 1H, J=7.5 Hz, J=7.8 Hz); 8.32 (s, br, 1H); 8.47 (d, 1H, J=4.8 Hz).

N-[(tert-butoxy)carbonylamino][7-chloro-4oxo-2-(pyrrolidinylcarbonyl)(3-hydroquinolyl)]-N-(2-pyridylmethyl)carboxamide.

To a stirred mixture of 7-chloro-4-oxo-2-(pyrrolidinylcarbonyl)hydroquinoline-3-carboxylic acid, Example 1, (17.5 g, 54.7 mmol) and dry THF (900 mL) under nitrogen was added CMC (35.7g, 81.2 mmol) in portions (25.0 g followed by 10.7 g after 10 minutes). After stirring the reaction mixture for an additional hour, a solution of (tert-butoxy)-N-[(2-pyridylmethyl)amino]carboxamide (16.5 g, 73.9 mmol) and THF (400 mL) was added and the mixture was vigorously stirred overnight. The reaction was monitored by TLC (10% methanol/DCM) and determined to be complete. To separate the precipitated solids, the reaction mixture was filtered and the collected solids were washed with THF. The filtrate and washings were combined and concentrated in vacuo. The filter cake was suspended in aqueous bicarbonate and brine solutions and extracted with DCM (3×300 mL). These extracts were combined with the previously concentrated organic extracts and were washed with bicarbonate, brine (3×) and dried over Na₂SO₄. The Na₂SO₄ was filtered off and the filtrate was concentrated under reduced pressure to provide a residue which was purified by flash chromatography on silica gel eluting with 5% iso-propanol/chloroform. After concentration of the desired fractions in vacuo the title compound was isolated as a light tan powder (24.3 g, 61% yield).

7-Chloro-4-hydroxy-2-(2-pyridylmethyl)-1,2,5,10-tetrahydropyridazino[4,5-b]quinoline-1,10-dione Methanesulfonate.

To a stirred mixture of N-[(tert-butoxy)carbonylamino][7-chloro-4-oxo-2-(pyrrolidinylcarbonyl)(3-hydroquinolyl)]-N-(2-pyridylmethyl)carboxamide (24.0 g, 0.045 mole) and dry TBF (800 mL) under nitrogen was added methanesulfonic acid (100 mL, 148 g, 1.54 moles) all at once. The mixture was stirred overnight and then filtered to separate the solids. The collected solids were successively washed with TEF (2×100 mL) and diethyl ether (2×100 mL). The filter cake (15.8 g) was then suspended in methanol (250 mL) and the resulting mixture sonicated for 30 minutes and then filtered. The collected solids were washed with methanol (2×100 mL) and diethyl ether (100 mL) and then dried at 35° C. in vacuo to give the title compound (12.1 g, 59%) as an orange powder, m.p. >300° C. ¹H NMR (300 MHz, DMSO-d⁶): δ 2.33 (s, 3H), 5.35 (s, 2H); 7.46 (d, 1H, J=8.7 Hz); 7.64 (d, 1H, J=7.8 Hz); 7.68 (dd, 1H, J=4.8 Hz, J=6.6 Hz); 8.02 (s, 1H); 8.14 (d, 1H, J=8.7 Hz); 8.19 (dd, 1H, J=6.6 Hz, J=7.8 Hz); 8.73 (d, 1H, J=4.8 Hz); 10.06 (s, br, 1H); 12.84 (s, br, 1H). Calc'd. for C₁₇H₁₁ClN₄O₃.CH₃SO₃H: C, 47.95; H, 3.35; N, 12.43 Found: C, 47.93; H, 3.42; N, 12.01

### Example 4

### 7-Chloro-4-hydroxy-2-benzo[d]furan-2-ylmethyl-1,2,5,10-tetrahydropyridazino[4,5-b]quinoline-1,10-dione

N-(1-aza-2-benzo[d]furan-2-ylvinyl)(tert-butoxy)carboxamide.

To a solution of benzofuran-2-carboxaldehyde (5.0 g, 34 mmol) in THF (200 mL) was added tert-butyl carbazate (4.5 g, 34 mmol) followed by concentrated HCl (10 drops) at room temperature with stirring. This reaction was stirred 24 h, at which time the THF was removed in vacuo and the resultant solid was triturated with hexanes and filtered to afford the title compound (9 g, 100%) as a white solid. ¹H NMR (300 MHz, DMSO-d₆): δ 11.12 (br s, 1H); 8.02 (s, 1H); 7.67 (d, J=7.6Hz, 1H); 7.62 (d, J=8.5Hz, 1H); 7.37 (dd, J=7.6, 7.6 Hz, 1H); 7.27 (dd, J=7.6, 7.6 Hz, 1H); 7.21 (s, 1H); 1.48 (s, 9H).

N-[(benzo[d]furan-2-ylmethyl)amino](tert-butoxy)carboxamide.

To a solution of N-(1-aza-2-benzo[d]furan-2-ylvinyl)(tert-butoxy)carboxamide (4.0 g, 15 mmol) in methanol (75 mL) was added sodium cyanoborohydride (7.2 g, 115 mmol) and acetic acid (10 mL). This mixture was heated to 65° C. for 4 h. TLC analysis (1:1, hexanes:ethyl acetate) showed starting material remained, and additional sodium cyanoborohydride (ca. 2 g) was added. After 2 more hours, no starting material remained and the reaction was cooled to room temperature and the methanol was removed in vacuo. The residue was dissolved in ethyl acetate and washed successively with saturated aqueous NaHCO₃, water and brine and then dried over Na₂SO₄. The mixture was filtered and concentrated to give the title compound (3.4 g, 13 mmol, 85%) as a white solid which was used in the following step without further purification. ¹H NMR (300 MHz, DMSO-d₆): δ 8.36 (s, 1H); 7.57 (d, J=7.0 Hz, 1H); 7.51 (d, J=7.7 Hz, 1H); 7.22 (m, 2H); 6.74 (s, 1H); 5.01 (br s, 1H); 4.00 (s, 2H); 1.37 (s, 9H).

N-{N-(benzo[d]furan-2-ylmethyl)[7-chloro-4-oxo-2-(pyrrolidinylcarbonyl)(3-hydroquinolyl)]carbonylamino}(tert-butoxy)carboxamide.

To a stirred slurry of 7-chloro-4oxo-2-(pyrrolidinylcarbonyl)hydroquinoline-3-carboxylic acid, Example 1, (4.1 g, 13 mmol) in THF (75 mL) was added 1-cyclohexyl-3-(2-morpholinoethyl)-carbodiimide metho-p-toluenesulfonate (10.8 g, 26 mmol). To this stirred canary-yellow mixture was added a solution of N-[(benzo[d]furan-2-ylmethyl)amino](tert-butoxy)carboxamide (3.3 g, 13 mmol) and N,N-dimethylaminopyridine (230 mg, 1.9 mmol) in THF (25 mL) with stirring. The resultant mixture was refluxed under N₂ for 4 h, then cooled and filtered. The filtrate was concentrated to give a solid yellow foam, which was chromatographed on silica gel (10% methanol in CH₂Cl₂) to afford the title compound as a pale yellow solid. This material was used in the following step without characterization.

7-Chloro-4-hydroxy-2-benzo[d]furan-2-ylmethyl-1,2,5,10-tetrahydropyridazino[4,5-b]quinoline-1,10-dione.

To a mixture of N-{N-(benzo[d]furan-2-ylmethyl)[7-chloro-4-oxo-2-(pyrrolidinyl-carbonyl)(3-hydroquinolyl)]-carbonylamino}(tert-butoxy)carboxamide (6.2 g, 11 mmol) in THF (150 mL) was added a room temperature solution of methanesulfonic acid (29 mL, 44 mmol) in TJF (70 mL). This solution was stirred overnight, after which time water (˜500 mL) was added to induce precipitation of the product A cream-colored solid was collected and rinsed with water and diethyl ether. This material was dried at 30° C. at 500 mTorr overnight to give the title compound as an off-white solid. ¹H NMR (300 MHz, DMSO-d₆): δ 12.74 (br s, 1H); 11.96 (br s, 1H); 8.15 (d, J=8.8 Hz, 1H); 8.04 (d, J=1.6 Hz, 1H); 7.59 (d, J=7.7 Hz, 1H): 7.53 (d, J=8.1 Hz, 1H); 7.44 (dd, J=2.0, 8.9 Hz, 1H); 7.25 (m, 2H); 6.84 (s, 1); 5.27 (s, 2H). Calc'd for C₂₀H₁₂N₃O₄Cl—0.1H₂O—0.3CH₃SO₃H: C, 57.45; H, 3.18; N, 9.90; Found: C, 57.61; H, 3.20; N, 9.91.

### Example 5

### 7-Chloro-4-hydroxy-2-(quinolin-4-ylmethyl)-1,2,5,10-tetrahydropyridazino[4,5-b]quinoline-1,10-dione

The title compound was synthesized by the method of Example 4 using quinoline-4-carboxaldehyde as the starting material. ¹H NMR (300 MHz, DMSO-d₆) δ 12.87 (br s, 1H); 12.10 (br s, 1H);9.16(d, J=5.4 Hz, 1H); 8.61 (d, J=8.4 Hz, 1H); 8.30 (d, J=8.4 Hz, 1H); 8.19-8.15 (m, 2H); 8.08 (d, J=1.8 Hz, 1H); 8.01 (dd, J=7.5, 7.8 Hz, 1H); 7.74 (d, J=5.4 Hz, 1H); 7.47 (dd, J=1.8, 8.7 Hz, 1H); 5.84 (s, 1H).

### Example 6

### 7-Chloro-4-hydroxy-2-(pyrazin-2-ylmethyl)-1,2,5,10-tetrahydropyridazino[4,5-b]quinoline-1,10-dione methanesulfonate

2-Chloromethylpyrazine.

2-Methylpyrazine (1.0 mL, 22 mmol) in carbon tetrachloride (80 mL) was treated with N-chlorosuccinimide (4.27 g, 31.5 mmol) and benzoyl peroxide (0.26 g, 1.1 mmol). The mixture was heated to reflux for 7 hours, and then cooled to room temperature. The solids were filtered through diatomaceous earth and washed with DCM. The filtrate was washed with aqueous sodium thiosulfate (sat., 1×), aqueous sodium bicarbonate (sat., 1×), water (1×), and aqueous sodium chloride (sat., 1×). The organic layer which contained 5-10% of the a,a,-dichlorinated material was dried over Na₂SO₄, filtered, concentrated under reduced pressure, and used directly in the following reaction. ¹H NMR (300 MHz, DMSO-d₆): δ 4.88 (s, 2H), 8.65-8.68 (m, 2H); 8.85 (s, 1H).

(tert-Butoxy)-N-[(pyrazin-2-ylmethyl)amino]carboxamide.

To a stirred solution of 2-chloromethylpyrazine (1.2 g, 93 mmol) and dry DMF (16 mL) was added tert-butylcarbazate (6.3 g, 48 mmol) and triethylamine (2.6 mL, 19 mmol) under nitrogen. The reaction mixture was then heated at 75° C. for 17 hours and cooled to room temperature. The reaction mixture was diluted with water and extracted with diethyl ether (5×). The combined ether extracts were washed with brine and dried over Na₂SO₄. The Na₂SO₄ was filtered off and the filtrate was concentrated under reduced pressure to give a brown oil (1 g). The oil was subjected to flash chromatography (silica gel, 2-5% gradient of methanol in DCM) to give the title compound as a waxy brown solid (1.64 g, 79%). ¹H NMR (300 MHz, DMSO-d₆, TFA shake): δ 1.44 (s, 9H); 4.53 (s, 2H); 8.71-8.78 (m, 2H); 8.81 (s, 1H).

N-[(tert-butoxy)carbonylamino][7-chloro-4-oxo-2-(pyrrolidinylcarbonyl)(3-hydroquinolyl)]-N-(pyrazin-2-ylmethyl)carboxamide.

To a stirred mixture of 7-chloro-4-oxo-2-(pyrrolidinylcarbonyl)hydroquinoline-3-carboxylic acid, Example 1, (2.3 g, 7.2 mmol) and dry THF (100 mL) under nitrogen was added CMC (4.56 g, 10.8 mmol). After stirring 20 minutes, the reaction mixture was treated with a solution of (tert-butoxy)-N-[(pyrazin-2-ylmethyl)amino]carboxamide (1.6 g, 7.1 mmol) and DMAP (46 mg, 0.4 mmol) in THF (10 mL). The reaction mixture was stirred at reflux for 2 days and cooled to room temperature. The reaction mixture was filtered and the filter cake was washed with THF. The combined filtrate and washes were concentrated to give a brown foam (4.9 g). The foam was subjected to flash chromatography (silica gel, 2% methanol/DCM) to give the title compound (3.1 g, 82%).

7-Chloro-4-hydroxy-2-(pyrazin-2-ylmethyl)-1,2,5,10-tetrahydropyridazino[4,5-b]quinoline-1,10-dione methanesulfonate.

To a stirred mixture of N-[(tert-butoxy)carbonylamino][7-chloro-4-oxo-2-(pyrrolidinylcarbonyl)(3-hydroquinolyl)]-N-(pyrazin-2-ylmethyl)carboxamide (3.1 g, 5.9 mmol) and dry THF (100 mL) under nitrogen was added methanesulfonic acid (13.5 mL, 0.21 mol). The mixture was stirred overnight, filtered, and the collected solid washed with THF. The solid was treated with methanol and the mixture sonicated for 1 h. The solid was again collected by filtration, washed with methanol, and dried at 50° C. in vacuo to give the title compound (2.0 g, 80%) as a white powder, m.p. 235-245° C. ¹H NMR (300 z, DMSO-d₆): δ 2.38 (CH₃CO₂H, s, 3H), 5.27 (s, 2H); 7.44 (dd, 1H, J=1.8, 8.7 Hz); 8.04 (d, 1H, J=1.8 Hz); 8.14 (d, 1H, J=8.7 Hz); 8.58 (dd, 2H, J=2.4, 7.5 Hz); 8.63 (s, 1H). Calc'd. for C₁₆H₁₀ClN₅O₃.CH₃SO₃H.H₂O: C, 43.46; H, 3.43; N, 14.90. Found: C, 43.28; H, 3.34; N, 15.17.

### Example 7

### 7-Chloro-4hydroxy-2-(5-isoxazolino)methyl-1,2,5,10-tetrahydropyridazino[4,5-b]quinoline-1,10-dione

N′-Isoxazol-5-ylmethyl-hydrazinecarboxylic Acid Tert-butyl Ester.

A mixture of 5-bromomethyl-isoxazole (1.62 g, 10 mmol), tert-butylcarbazate (5.29 g, 40 mmol) and sodium carbonate (2.76 g, 20 mmol) in DMF (25 mL) was heated to 80° C. under a nitrogen atmosphere for 6 hours. The mixture was cooled and partitioned between ethyl acetate (100 mL) and water (200 mL). The organic layer was washed with brine (3×50 mL) and dried over MgSO₄. The solvent was removed by rotary evaporation. The residual DMF and excess tert-butylcarbazate was removed by vacuum distillation (50 mTorr, 80° C.). The residue was subjected to chromatography (silica gel, 1/1 ethyl acetate/hexane) to give the title compound as a white solid (1.01 g, 49%). ¹H NMR (300 MHz, DMSO-d₆): δ 1.38 (s, 9H); 4.01 (s, 2H); 5.13 (bs, 1H); 6.38 (s, 1H); 8.38 (s, 1H); 8.48 (s, 1H).

N′-[7-chloro-4-hydroxy-2-(pyrrolidine-1-carbonyl)quinoline-3-carboxyl]-N′-isoxazol-5-ylmethyl-hydrazinecarboxylic Acid Tert-butyl Ester.

To a stirred slurry of 7-chloro-4-oxo-2-(pyrrolidinylcarbonyl)hydroquinoline-3-carboxylic acid, Example 1, (1.51 g, 4.7 mmol) in THF (50 mL) was added CMC (4.24 g, 10 mmol) and the reaction was stirred for five minutes. To this mixture was added a solution of N′-isoxazol-5-ylmethyl-hydrazinecaboxylic acid tert-butyl ester (1.0 g, 4.7 mmol) and DMAP (0.06 g, 0.5 mmol) in THF (10 mL). The mixture was heated to reflux for 1.5 hours then allow to stand at room temperature for 16 hours. The solids were filtered off and washed with DCM (2×50 mL). The combined filtrate was evaporated to dryness by rotary evaporation. The residual solid was subjected to chromatography (silica gel, 1/9 methanol/DCM) to give the title compound as an off-white foam (2.09 g, 86%). MS (CI) m/z 514/516.

7-Chloro-4-hydroxy-2-(5-isoxazolino)methyl-1,2,5,10-tetrahydropyridazino[4,5-b]quinoline-1,10-dione.

To a solution of N′-[7-chloro-4-hydroxy-2-(pyrrolidine-1-carbonyl)-quinoline-3-carboxyl]-N′-isoxazol-5-ylmethyl-hydrazinecarboxylic acid tert-butyl ester (1.0 g, 1.94 mmol) in THF (50 mL) was added methanesulfonic acid (5.2 mL). After 18 hours the solvent was removed by rotary evaporation and the product was precipitated by the addition of water (100 mL). The solid was collected by vacuum filtration and washed with water (2×50 mL) then dried in vacuo (500 mTorr, 30° C.) for 16 hours. The solid was suspended in diethyl ether (45 mL) and methanol (5 mL) and sonicated for 10 minutes. The solid was collected by vacuum filtration, washed with diethyl ether (2×30 mL), and dried in vacuo (500 mTorr, 30° C.) for 18 hours. This gave the title compound as a yellow solid (0.54 g, 81%). ¹¹H NMR (300 MHz, DMSO-d₆): δ 5.27 (s, 2H); 6.44 (s, 1H); 7.45 (dd, 1H, Jₒ=8.7 Hz, Jₘ=1.8 Hz); 8.03 (d, 1H, Jₘ=1.8 Hz); 8.15 (d, 1H, J.ₒ=8.7 Hz); 8.53 (s, 1H, Jₘ=1.8 Hz); 11.99 (s, 1H); 12.82 (s, 1H). Calc'd. for C₁₅H₉ClN₄O₄.0.4H₂O: C, 51.20; H, 2.81; N, 15.92; Found: C, 51.48-51.33; H, 2.79-2.77; N, 15.60-15.57.

### Example 8

### 7-Chloro-4-hydroxy-2-(pyrimidin-2-ylmethyl)-1,2,5,10-tetrahydropyridazino[4,5-b]quinoline-1,10-dione

The title compound was synthesized by the method of Example 7 using 4-chloromethylpyrimidine, prepared as described by Barnes, J. H., et al Eur. J. Med. Chem. Chim. Ther. 1988, 23, 211-216, as the starting material. ¹H NMR (300 MHz, DMSO-d₆) δ 12.77 (br s, 1H); 9.12 (d, J=1.2 Hz, 1 H); 8.74 (d, J=5.4 Hz, 1 H); 8.15 (d, J=8.4 Hz, 1 H); 8.05 (d, J=1.8 Hz, 1 H); 7.45 (dd, J=2.1, 8.7 Hz, 1H);7.40(d, J=5.1Hz, 1 H); 5.20 (s, 2 H).

### Example 9

### 7-Chloro-4hydroxy-2-(furan-2-ylmethyl)-1 2,5,10-tetrahydropyridazino[4,5-b]quinoline-1,10-dione

N-1-aza-2-(2-furanyl)vinyl)(tert-butoxy)carboxamide.

To a stirred slurry of tert-butylcarbazate (131.5 g, 0.99 mol) in hexane (1000 m-mL) was added 2-furaldehyde (91.9 g, 0.95 mol). The slurry was refluxed for 2.5 h, and then cooled to room temperature. The resultant tan solid was filtered, dried and used without further purification in the following reaction (200 g, 99%). ¹H NMR (300 MHz, DMSO-d₆): δ 1.52 (s, 9H); 6.45 (dd, 1H, J=3.3, 1.2 Hz); 6.71 (d, 1H, J=3.3 Hz); 7.47 (d, 1H, J=1.2 Hz); 7.91 (s, 1H).

(tert-Butoxy)-N-[(2-furanylmethyl)amino]carboxamide.

A one liter, three-neck round bottom flask was equipped with an addition funnel, nitrogen inlet and an overhead mechanical stirrer. The apparatus was dried in vacuo and flushed with a steady stream of nitrogen gas. The flask was charged with lithium aluminum hydride (7.75 g, 0.20 mol) and THF (30 mL). N-1-Aza-2-(2-furanyl)vinyl)(tert-butoxy)carboxamide (20 g, 0.095 mol) was dissolved in THF (250 mL) and then slowly added to the stirred lithium aluminum hydride suspension over a 30 minute period. Any residual material remaining in the addition funnel was washed into the flask by rinsing with THF (2×30 mL). The reaction was stirred overnight, cooled with an ice bath and then carefully quenched with a saturated aqueous solution of Na₂SO₄. The resulting mixture was filtered and the collected solids washed with THF. The combined filtrate and washes were concentrated to an oil, which was stirred for 18 hours with hexanes (ca. 600 mL). The resulting mixture was filtered and the filtrate concentrated to give the desired material as a yellow oil (10.0 g, 50%). ¹H NMR (300 MHz, DMSO-d₆): δ 1.46 (s, 9H); 3.99 (d, 2H, J=4.9 Hz); 4.21 (br s, 1H); 6.17 (br s, 1H); 6.24 (d, 1H, J=3.0 Hz); 6.32 (dd, 1H, J=3.0, 1.2 Hz); 7.38 (d, 1H, J=1.2 Hz).

(+/−)-N-[(tert-Butoxy)carbonylamino][7-chloro-4-oxo-2-(pyrrolidinylcarbonyl)(3-hydroquinolyl]-N-(2-furanylmethyl)carboxamide.

To a stirred slurry of 7-chloro-4-oxo-2-(pyrrolidinylcarbonyl)hydroquinoline-3-carboxylic acid, Example 1, (26.99 g, 84.3 mmol) in THF (1300 mL) was added di-iso-propylcarbodiimide (13.94 g, 110 mmol) and the reaction was stirred for ten minutes. To this mixture was added dropwise a solution of (tert-butoxy)-N-[(2-furanylmethyl)amino]carboxamide (22.9 g, 103 mmol) in THF (200 mL). After stirring the reaction for 18 hours, it was concentrated in vacuo to a brown tar which was triturated with chloroform. The resulting mixture was filtered, washed with chloroform and dried. This material was used without further purification in the following reaction (mass of material approximately 30 g).

7-Chloro-4-hydroxy-2-(2-furanylmethyl)-1,2,5,10-tetrahydropyridazino[4,5-b]quinoline-1,10-dione.

To a stirred solution of (+/−)-N-[(tert-butoxy)carbonylamino][7-chloro-4-oxo-2-(pyrrolidinylcarbonyl)(3-hydroquinolyl]-N-(2-furanylmethyl)carboxamide (29.00 g, 56.3 mmol) in THF (1000 mL) was added methanesulfonic acid (118 mL) and the reaction was stirred for 18 hours at room temperature. The reaction mixture was poured on 3 liters of ice-water and the resulting mixture was filtered to give an off-white solid. This material was dissolved in hot THF (ca. 1 liter) and then concentrated to half-volume. The resultant slurry was poured on ice-water (2 liters) and after twenty minutes the mixture was filtered. The collected material was dried to give the title compound as a white solid (14.4 g, 74%; m.p.>265° C.). ¹H NMR (300 MHz, DMSO-d₆): δ 5.08 (s, 2H); 6.37 (d, 1H, J=3.0 Hz); 6.42 (dd, 1H, J=3.0, 1.5 Hz); 7.43 (d, 1H, J=7.8 Hz); 7.54 (s, 1H); 8.00 (d, 1H, J=1.5 Hz); 8.14 (d, 1H, J=8.7Hz). Calc'd. for C₁₆H₁₀ClN₃O₄: C, 55.91; H, 2.93; N, 12.23 Found: C, 56.10; H, 2.98; N, 12.03.

### Example 10

### 7-Chloro-4-hydroxy-2-(furan-3-ylmethyl)-1,2,5,10-tetrahydropyridazino[4,5-b]quinoline-1,10-dione

N-1-Aza-2-(3-furyl)vinyl)(tert-butoxy)carboxamide.

To a solution of furan-3-carboxaldehyde (2.0 g, 21 mmol) in THF (100 mL) was added tert-butyl carbazate (2.8 g, 21 mmol) and conc. HCl (5 drops). This solution was stirred for 6 h and concentrated. The resultant solid was triturated with hexanes to afford the title compound (4.2 g, 20 mmol, 97%) as a peach-colored solid. ¹H NMR (300 MHz, DMSO-d₆): δ 10.77 (br s, 1H); 8.02 (s, 1H); 7.94 (s, 1H); 7.71 (dd, J=1.5, 1.5 Hz, 1H); 6.77 (d, J=1.5 Hz, 1H); 1.45 (s, 9H).

(tert-Butoxy)-N-[(3-furylmethyl)amino]carboxamide.

To a solution of N-1-aza-2-(3-furyl)vinyl)(tert-butoxy)carboxamide (2.0 g, 9.5 mmol) in methanol (50 mL) was added sodium cyanoborohydride (3.0 g, 48 mmol) and acetic acid (6 mL). This solution was heated to 65° C. for 1 h. The mixture was then cooled to room temperature, quenched with water and the methanol was removed in vacuo. The residue was taken up in ethyl acetate and rinsed with saturated sodium bicarbonate, water and brine and then dried over Na₂SO₄. This was filtered and concentrated and the residue was purified by silica gel chromatography (4:1 hexanes:ethyl acetate) to afford the title compound (930 mg, 4.4 mmol, 46%) as a clear oil. ¹H NMR (300 MHz, DMSO-d₆): δ 8.26 (br s, 1H); 7.58 (dd, J=1.5, 1.5 Hz, 1H); 7.53 (s, 1H); 6.42 (d, J=1.2 Hz, 1H); 4.64 (br s, 1H); 3.69 (s, 2H); 1.39 (s, 9H).

(tert-Butoxy)-N-{[7-chloro-4-oxo-2-(pyrrolidinylcarbonyl)(3-hydroquinolyl)]-N-3-(furylmethyl)carbonylamino}carboxamide.

To a stirred slurry of 7-chloro-4-oxo-2-(pyrrolidinylcarbonyl)hydroquinoline-3-carboxylic acid, Example 1, (1.4 g, 4.4 mmol) in THF (25 mL) was added CMC (3.7 g, 8.8 mmol). To this stirred canary yellow mixture was added a solution of (tert-butoxy)-N-[(3-furylmethyl)amino]carboxamide (930 mg, 4.4 mmol) and N,N-dimethylaminopyridine (80 mg, 660 μmol) in THF (20 mL) with stirring. The resultant mixture was refluxed under N₂ for 3 h, then cooled and filtered. The filtrate was concentrated to give a solid yellow foam, which was chromatographed on silica gel (10% methanol-CH₂Cl₂) to afford the title compound (2.1 g, 4.1 mmol, 93%) as a pale yellow solid. This material was used directly in the following step.

7-Chloro-4-hydroxy-2-(furan-3-ylmethyl)-1,2,5,10-tetrahydropyridazino[4,5-b]quinoline-1,10-dione.

To a mixture of (tert-butoxy)-N-{[7-chloro-4-oxo-2-(pyrrolidinylcarbonyl)(3-hydroquinolyl)]-N-3-(furylmethyl)carbonylamino}carboxamide (1.4 g, 2.8 mmol) in THF (30 mL) was added a room temperature solution of methanesulfonic acid (7.2 mL, 110 mmol) in THF (25 mL). This solution was stirred overnight, at which time water was added to induce precipitation of the product. The solid was collected, rinsed with water and diethyl ether and sonicated for 15 min in 50 mL of a 10% methanol in diethyl ether solution. The resultant yellow solid was collected, rinsed with diethyl ether and dried at 30° C. and 50 mTorr for 3 h to afford the title compound (750 mg, 2.1 mmol, 75 %) as a pale yellow solid. ¹H NMR (300 MHz, DMSO-d₆): δ 12.63 (br s, 1H); 11.91 (s, 1H); 8.13 (d, J=8.7 Hz, 1H); 8.02 (d, J=1.5 Hz, 1H); 7.65 (s, 1H); 7.61 (d, J=1.5 Hz, 1H); 7.43 (dd, J=1.8, 8.7 Hz, 1H); 6.46 (s, 1H); 4.92 (s, 2H). Calc'd for C₁₆H₁₀N₃O₄Cl.0.1 H₂O: C, 55.62; H, 2.98; N, 12.16; Found: C, 55.67; H, 3.15; N, 11.77.

### Example 11

### 7-Chloro-4-hydroxy-2-(thien-2-ylmethyl)-1,2,5,10-tetrahydropyridazino[4,5-b]quinoline-1,10dione

The title compound was synthesized by the method of Example 4 using thiophene-2-carboxaldehyde as the starting material. ¹H NMR (300 MHz, DMSO-d₆) δ 12.71 (br s, 1H); 11.91 (s, 1H); 8.14 (d, J=8.7 Hz, 1H); 8.02 (s, 1H); 7.42-7.45 (m, 2H); 7.10 (d, J=3.0 Hz, 1 H); 6.98 (dd, J=3.6, 5.1 Hz, 1 H); 5.23 (s, 2H).

### Example 12

### 7-Chloro-4-hydroxy-2-(thien-3-ylmethyl)-1,2,5,10-tetrahydropyridazino[4,5-b]quinoline-1,10-dione

The title compound was synthesized by the method of Example 4 using thiophene-3-carboxaldehyde as the starting material. ¹H NMR (300 MHz, DMSO-d₆) δ 12.64 (br s, 1H); 11.92 (br s, 1 H); 8.14 (d, J=8.7, 1 H); 8.02 (d, J=1.5 Hz, 1 H); 7.48-7.54 (m, 1 H); 7.43 (dd, J=1.2, 8.7, 1 H); 7.37 (s, 1 H); 7.08 (d, J=4.5 Hz, 1 H); 5.08 (s, 2 H).

### Example 13

### 7-Chloro-4-hydroxy-2-(benzo[b]thien-2-ylmethyl)-1,2,5,10-tetrahydropyridazino[4,5-b]quinoline-1,10-dione

Benzo[b]thiophene-2-carbaldehyde.

To a solution of benzo[b]thiophene (10 g, 74.5 mmole ) in dry THF (12 mL) at −78° C. was added 65 mL of 1.6M n-butyl lithium in hexanes. Ten minutes later DMF (23 mL, 298 mmol) was added. The reaction was warmed to room temperature and then refluxed for 3 hours. The THP was evaporated and the residue was poured into 1 N HCl and ice. The acidic solution was extracted with diethyl ether (2×). The combined ether extract was washed with 1 N HCl (3×), saturated NaHCO₃ (1×), brine (1×), and then dried with MgSO₄. The MgSO₄ was filtered off and the filtrate concentrated to an oil which was treated with NaHSO₃. The solid that formed was collected, treated with aqueous NaHCO₃, then extracted with DCM. The DCM solution was dried over MgSO₄ and evaporated to give the title compound as a yellow oil (3.2 g, 26% yield). ¹H NMR (300 MHz, CDCl₃): δ 7.44 (dd, 1H, J=6.9, 7.2 Hz); 7.51 (dd, 1H, J=6.9, 8.1 Hz); 7.91 (d, 1H, J=8.1 Hz ); 7.95 (d, 1H, J=7.8 Hz); 8.04 (s, 1H); 10.12 (s, 1H).

(tert-Butoxy)-N-(1-aza-2-benzo[b]thien-2-ylvinyl)carboxamide.

To a stirred solution of benzo[b]thiophene-2-carbaldehyde (3.2 g, 19.7 mmol) and tert-butylcarbazate (2.6 g, 19.7 mmol) in ethanol (20 mL) was added 3 drops of concentrated HCl. After 30 minutes the mixture was filtered, the solid washed with diethyl ether, and dried in vacuo to give the title compound (3.8 g, 70% yield). ¹H NMR (300 MHz, CDCl₃): δ 1.54 (s, 9H); 7.30-7.36 (m, 2H); 7.40 (s, 1H); 7.71-7.74 (m, 1H); 7.78-7.81 (m, 1H); 7.89 (s, 1H); 8.31 (br s, 1H).

(tert-Butoxy)-N-[(benzo[b]thien-2-ylmethyl)amino]carboxamide.

To a slurry of (tert-butoxy)-N-(1-aza-2-benzo[b]thien-2-ylvinyl)carboxamide (1.8 g.. 6.5 mmol) in THF (6 mL) was added sodium cyanoborohydride (0.75 g, 11.9 mmol). A solution of p-toluene sulfonic acid (1.86g, 9.8 mmol) in THF (6 mL) was added dropwise, After stirring overnight, the reaction was diluted with ethyl acetate and washed with saturated NaHCO₃ (1×) and saturated NaCl (1×). The ethyl acetate solution was dried over K₂CO₃. The K₂CO₃ was filtered off and the filtrate was concentrated under reduced pressure. The resulting solid was treated with saturated NaHCO₃ overnight and extracted with DCM. The DCM solution was dried over Na₂SO₄. The Na₂SO₄ was filtered off and the filtrate concentrated to give the title compound as a white solid (1.7 g, 76% yield). ¹H NMR (300 MHz, CDCl₃): δ 1.44 (s, 9H); 4.68 (s, 2H); 7.36-7.45 (m, 2H); 7.45 (s, 1H); 7.78-7.86 (m, 2H).

N′-Benzo[b]thien-2-ylmethyl-N′-[7-chlor-4-oxo-2-(pyrrolidine-1-carbonyl)-1,4-dihydroquinoline-3-carbonyl]hydrazinecarboxylic Acid Tert-butyl Ester.

To a stirred mixture of 7-chloro-4-oxo-2-(pyrrolidinylcarbonyl)hydroquinoline-3-carboxylic acid, Example 1, (1.59 g, 5.0 mmol) and dry THF (60 mL) under nitrogen was added CMC (3.19 g, 7.0 mmol). This was followed by a solution of (tert-butoxy)-N-[(benzo[b]thien-2-ylmethyl)amino]carboxamide (1.37 g, 5.0 mmol) and dimethylaminopyridine (27.8 mg, 0.21 mmol) in THF (15 mL). The reaction was heated at reflux overnight and the mixture was filtered. The concentrated filtrate was purified by chromatography (MeOH/CH₂Cl₂, 5/95, v/v) to give the title compound as a yellow solid (771 mg, 24% yield).

7-Chloro-4-hydroxy-2-(benzo[b]thien-2-ylmethyl)-1,2,5,10-tetrahydropvridazino[4,5-b]quinoline-1.10-dione.

To a stirred mixture of N′-benzo[b]thien-2-ylmethyl-N′-[7-chlor-4-oxo-2-(pyrrolidine-1-carbonyl)-1,4-dihydroquinoline-3-carbonyl]hydrazinecarboxylic acid tert-butyl ester (760 mg, 1.3 mmol) and dry THF (16 mL) at 0° C. under nitrogen was added methanesulfonic acid (3.0 mL, 4.44 g, 46.2 mmol). After stirring overnight, the THP was evaporated and the residue cooled in an ice bath. Water was added and the resulting precipitate was collected, sonicated with methanol, and dried in vacuo to give the title compound as an off-white solid (470 mg, 88% yield), m.p. >300° C. ¹H NMR (300 MHz, DMSO-d₆): δ 5.38 (s, 2H); 7.30-7.38 (m, 2H,); 7.40 (s, 1H); 7.44 (d, 1H, J=8.7 Hz); 7.90 (d, 1H, J=7.2 Hz); 7.82 (d, 1H, 7.2 Hz); 8.06 (s, 1H); 8.15 (d, 1H, J=8.7 Hz). Calc'd. for C₂₀H₁₂ClN₃O₃S: C, 58.61; H, 2.95; N, 10.25. Found: C, 58.42; H, 3.19; N, 10.20.

### Example 14

### 7-Chloro-4-hydroxy-2-(1,3-thiazo-2-ylmethyl)-1,2,5,10-tetrahydropyridazino[4,5-b]quinoline-1,10-dione

(tert-Butoxy)-N-[(1,3-thiazol-2-ylmethyl)amino]carboxamide.

To a stirred solution of thiazole-2-carbaldehyde (0.95 g, 8.42 mmol) and tert-butylcarbazate (1.17 g, 8.87 mmol) in ethanol (15 mL) was added 1.10 mL of glacial acetic acid, followed by sodium cyanoborohydride (2.18 g, 34.7 mmol). The reaction mixture was heated at 50° C. and stirred for 72 hours. The reaction was quenched with 2 N NaOH (30 mL) and the resulting solution was extracted with ethyl acetate (3×30 mL). The combined organic layers were washed with brine (40 mL), then dried over Na₂SO₄. The Na₂SO₄ was filtered off and the filtrate was purified by chromatography (ethyl acetate:DCM, 25:75, v:v) over silica gel to give the title compound, (0.62 g, 32% yield) as an off-white solid. ¹H NMR (300 MHz, DMSO-d₆): δ 1.38 (s, 9H); 4.15 (d, 2H, J=3.9 Hz); 5.34 (d, 1H, J=3.9 Hz); 7.63 (d, 1H, J=3.3 Hz); 7.70 (d, 1H, J=3.3 Hz); 8.42 (br s, 1H).

N-[(tert-Butoxy)carbonylamino][7-chloro-4-oxo-2-(pyrrolidinylcarbonyl)(3-hydroquinolyl)]-N-(1,3-thiazol-2-ylmethyl)carboxamide.

To a stirred mixture of 7-chloro-4-oxo-2-(pyrrolidinylcarbonyl)hydroquinoline-3-carboxylic acid, Example 1, (0.90 g, 2.80 mmol) and dry THF (60 mL) under nitrogen was added CMC (1.45 g, 3.42 mmol). This was followed by a solution of (tert-butoxy)-N-[(1,3-thiazol-2-ylmethyl)amino]carboxamide (0.5 g, 2.20 mmol) and dimethylaminopyridine (79.1 mg, 0.65 mmol) in THF (15 mL). The reaction was heated at reflux overnight and, after cooling, the reaction mixture was filtered. The concentrated filtrate was purified by chromatography (MeOH:CH₂Cl_{2, 5:95}, v:v) over silica gel to give the title compound as a yellow solid (0.97 g, 83% yield).

7-Chloro-4-hydroxy-2-(1,3-thiazo-2-ylmethyl)-1,2,5,10-tetrahydropyridazino[4,5-b]quinoline-1,10-dione.

To a stirred mixture of N-[(tert-butoxy)carbonylamino][7-chloro-4-oxo-2-(pyrrolidinylcarbonyl)(3-hydroquinolyl)]-N-(1,3-thiazol-2-ylmethyl)carboxamide (760 mg, 1.3 mmol) and dry THF (40 mL) at 0° C. under nitrogen was added methanesulfonic acid (5.4 mL, 7.99 g, 83.2 mmol). After stirring overnight, the THF was evaporated and the residue cooled in an ice bath. Water was added and the resulting precipitate was collected, sonicated with methanol. The solid was collected by suction filtration and dried in vacuo to give the title compound as an off-white solid (648 mg, 78% yield), mp>300° C. ¹H NMR (300 MHz, DMSO-d₆): δ 5.38 (s, 2H); 7.45 (d, 1H, J=8.7 Hz); 7.70 (d, 1H, J=3.3); 7.76 (d, 1H, J=3.3Hz); 8.04 (s, 1H); 8.15 (d, 1H, 8.7 Hz). Calc'd. for C₁₅H₁₀ClN₄O₃S.H₂O.H₃CSO₃H: C, 40.38; H, 3.39; N, 11.77. Found: C, 40.63; H, 2.98; N, 11.39.

### Example 15

### 7-Chloro-4-hydroxy-2-(imidazol-2-ylmethyl)-1,2,5,10-tetrahydropyridazino[4,5-b]quinoline-1,10-dione

N-(1-Aza-2-imidazol-2-ylvinyl)(tert-butoxy)carboxamide.

2-Imidazolecarboxaldehyde (10.2 g, 106 mmol) was dissolved in THF. To this was added tert-butylcarbazate, followed by two drops of concentrated hydrochloric acid. The reaction was stirred overnight, concentrated and triturated with hexanes to give the title compound as a white solid (22 g, 99%). ¹H NMR (300 MHz, DMSO-d₆): δ 1.47 (s, 9H); 7.08 (s, 2H); 7.92 (s, 1H); 10.93 (br s, 1H); 12.58 (br s, 1H).

(tert-Butoxy)-N-[(imidazol-2-ylmethyl)amino]carboxamide.

A mixture of 10% palladium on carbon (0.50 g) and N-(1-aza-2-imidazol-2-ylvinyl)(tert-butoxy)carboxamide (3.0 g, 14.0 mmol) in methanol (40 mL) and concentrated hydrochloric acid (1.15 mL, 14.0 mmol) was hydrogenated (40 psi) at room temperature for 18 hours. The reaction was filtered through diatomaceous earth and the filtrate evaporated under reduced pressure to give an oil. The oil was neutralized by the addition of sodium hydroxide (5 N, 2.8 mL) and then diluted with ethyl acetate (80 mL). The ethyl acetate layer was washed with water (1×20 mL) and sodium chloride (sat. aqueous, 1×20 mL) and then dried over Na₂SO₄. The ethyl acetate was removed to give the title compound as an oil (2.42 g, 80%). ¹H NMR (300 MHz, DMSO-d₆): δ 1.36 (s, 9H); 3.84 (d, 2H, J=4.2 Hz); 4.82 (br s, 1H); 6.91 (s, 2H); 8.25 (br s, 1H); 11.84 (br s, 1H).

N-[(tert-Butoxy)carbonylamino][7-chloro-4-oxo-2-(pyrrolidinylcarbonyl)(3-hydroquinolyl]-N-(imidazol-2-ylmethyl)carboxamide.

A mixture of 7-chloro-4-oxo-2-(pyrrolidinylcarbonyl)hydroquinoline-3-carboxylic acid, Example 1, (2.42 g, 7.54 mmol), (tert-butoxy)-N-[(imidazol-2-ylmethyl)amino]carboxamide (2.0 g, 9.43 mmol), and CMC (4.14 g, 9.80 mmol) in THF(50 mL, dry) was refluxed for 18 hours. The reaction was filtered and the solids were collected. The solids were washed with water and then diethyl ether. This material was dried in vacuo to give the title compound as a white solid (1.0 g, 25%).

7-Chloro-4-hydroxy-2-(imidazol-2-ylmethyl)-1,2,5,10tetrahydropyridazino[4,5-b]quinoline-1,10-dione.

To a stirred solution of N-[(tert-butoxy)carbonylamino][7-chloro-4-oxo-2-(pyrrolidinylcarbonyl)(3-hydroquinolyl]-N-(imidazol-2-ylmethyl)carboxamide (1.0 g, 1.94 mmol) in THF (30 mL) was added methanesulfonic acid (5 mL) and the reaction was stirred overnight. The volatiles were removed in vacuo and to the residual oil was added diethyl ether (200 mL). The mixture was stirred for 10 minutes and then allowed to settle into two layers, an etheral layer and layer of brown oil. The ether was decanted away and to the brown oil was added water (5 mL). After a short time, a precipitate formed and was collected by vacuum filtration. The precipitate was washed with a diethyl ether (3×20 mL) and then sonicated in 20 mL of 10/1 diethyl ether/methyl alcohol for fifteen minutes. The material was filtered, washed with diethyl ether and dried in vacuo to give the title compound (0.24 g, 25%). ¹H NMR (300 MHz, DMSO-d₆): δ 2.31 (CH₃SO₃H); 5.41 (s, 2H); 7.42 (d, 1H, J=8.7 Hz); 7.67 (s, 2H); 7.99 (s, 1H); 8.15 (d, 1H, J=8.7 Hz); 12.7 (br s, 1H); 11.98 (br s); 12.93 (br s, 1H); 14.28 (br s, 1H). Calc'd. for C₁₅H₁₀ClN₄O₃.1.6CH₃SO₃H: C, 40.08; H, 3.32; N, 14.08; Found: C, 40.37; H, 3.12; N, 14.34.

Tests for Biological Function:

Test A: Inhibition of Binding of [³H]-MDL105,519:

Binding of compounds to the NMDA receptor glycine site may be assessed by measuring the ability of test compounds to inhibit the binding of tritiated MDL105,519 to brain membranes bearing the receptor.

Rat Brain Membranes:

The rat brain membranes used in the experiments were obtained from Analytical Biological Services Inc., and were prepared substantially in accordance with the method of B. M. Baron et al., J. Pharmacol. Exp. Ther. 250, 162 (1989). Briefly, fresh brain tissue including cerebral cortex and hippocampus from male Sprague Dawley rats was homogenized in 0.32 M sucrose and centrifuged at low speed to separate cellular membranes from other cellular components. The membranes were then washed 3 times using deionized water, followed by treatment with 0.04% Triton X-100. Finally, membranes were washed six times in 50 mM Tris citrate buffer, pH 7.4, and frozen at −80° C. until use.

[³H]MDL105,519 (72 Ci/mmol) was purchased from Amersham. Cold MDL105,519 was purchased from Sigma/RBI. Binding assays were performed substantially in accordance with the protocol of B. M. Baron et al., J. Pharmacol. Exp. Ther. 279, 62 (1996), as follows. On the day of the experiment, brain membranes were thawed at room temperature and suspended in 50 mM tris acetate buffer, pH 7.4 (“TAB”). Seventy-five micro grams per milliliter protein (by using the BioRad dye) were used for competition binding. The experiments were carried out using 96-well plates. Membranes were incubated with 20 μL of compounds of various concentrations and 1.2 nM [³H]MDL105,519 for 30 minutes at room temperature in a total volume of 250 μL. Non specific binding was determined by using 100 μM of unlabeled MDL105,519. The unlabeled MDL105,519 and compounds were dissolved as 12.5 mM stock solutions in DMSO. Final DMSO concentration in each well was kept below 1%, which concentration was found not to alter the binding results. After incubation, unbound [³H]MDL105,519 was removed by filtration onto GF/B Unifilter plates using a Packard harvester. Filters were washed four times with ice cold TAB (total of 1.2 mL buffer). The plates were dried overnight at room temperature and bound radioactivity was measured on a Packard TopCount after the addition of 45 μL per well of the MICROSCINT O.

Human Brain Membranes:

Human brain membranes were obtained from Analytical Biological Services Inc., and assays were performed as described for rat membranes.

Data Analysis:

Data was analyzed using a Microsoft Excel spreadsheet and GraphPad Prizm software and potency of compounds is expressed as the Ki (nM).

Test B: Formalin test:

The Formalin test is an assay that assesses the capacity of a compound to inhibit formalin-induced nociceptive behaviors in rats (D. Dubuisson, et al., Pain 4, 161-174 (1977); H. Wheeler-Aceto et al., Psychopharmacology 104, 35-44 (1991); T. J. Coderre, et al., Pain 54, 43-50 (1993)). In the test, two distinctive phases of formalin-induced behaviors are observed. A first phase response, caused by acute nociception to the noxious chemical (formalin) injected into the paw, occurs between zero and five minutes. A quiescent period of 5 to 15 min post injection follows. After the quiescent period a second phase response, caused by sensitization of the central neurons in the dorsal horn, occurs after 15 minutes and lasts up to 60 minutes. Sensitization of the central neurons in the spine augments a noxious afferent input and causes a stronger pain barrage to be transmitted to the brain. Therefore, inhibition of the second phase response indicates a central mechanism of drug action.

The procedure for the formalin test is as follows: male rats are placed in a plexiglass chamber and observed for 30-45 min. to observe their baseline activity. Animals are either pretreated with vehicle or with different doses of a test compound. Animals are dosed with vehicle or test compound three hours prior to injection of 0.05 mL of sterile 1% formalin under the dorsal skin of a hind paw. The number of paw flinches (responses) during the first phase (0-5 min.) and the second phase (20-35 min.) are scored and recorded. Flinch response is compared with the mean score of a saline control group and calculated as percentage inhibition. The ED₅₀ is the dose of compound which produces 50% inhibition of nociceptive response in the first or second phase response. First phase responses may be inhibited by compounds that act peripherally and by compounds that act centrally. Second phase response are inhibited by centrally active compounds.

% inhibition of nociceptive response=100×(number of responses in vehicle group−number of responses in compound group)/(number of responses in vehicle group)

Student's t-test was used for statistical analysis to determine the significance of compound effects. Data are reported as a dose that yielded a % inhibition of a response.

Test C: Neuropathic Pain Model (Chronic Constriction Injury):

The anti-hyperalgesic properties of a compound may be tested with the Chronic Constriction Injury (“CCI”) model. The test is a model for neuropathic pain associated with nerve injuries that can arise directly from trauma and compression, or indirectly from a wide range of diseases such as infection, cancer, metabolic conditions, toxins, nutritional deficiencies, immunological dysfunction, and musculoskeletal changes. In the model a unilateral peripheral hyperalgesia is produced in rats by nerve ligation (G. J. Bennett, et al., Pain 33, 87-107 (1988)).

Procedurally, Sprague-Dawley rats (250-350 g) are anesthetized with sodium pentobarbital and the common sciatic nerve is exposed at the level of the mid thigh by blunt dissection through the biceps femoris. A section of nerve (about 7 mm), proximal to the sciatic trifucation, is freed of tissue and ligated at four positions with chromic gut suture. The suture is tied with about 1 mm spacing between ligatures. The incision is closed in layers and the animals are allowed to recuperate. Thermal hyperalgesia is measured using a paw-withdrawal test (K. Hargreaves, et al., Pain 32, 77-88 (1988)). To perform the test, animals are habituated on an elevated glass floor. A radiant heat source is aimed at the mid-plantar hindpaw (sciatic nerve territory) through the glass floor with a 20 second cut-off used to prevent injury to the skin. The latencies for the withdrawal reflex in both hind paws are recorded.

Injured paws with ligated nerves show shorter paw withdrawal latencies compared to the uninjured or sham operated paws. Responses to test compounds are evaluated at different times after oral administration to determine the onset and duration of compound effect. When performing the test, groups of CCI rats receive either vehicle or the test compound orally three times daily for 5 days. Paw withdrawal latencies are measured each day 10 min before and 2 or 3 hr. after the first daily dose. Compound efficacy is expressed as mean percentage decrease of hyperalgesia compared to that of vehicle-treated animals, calculated as follows:

(Mean of vehicle group−Mean of compound group)/(Mean of vehicle group)×100

Data analysis was performed by the multiple means comparison test (Dunnett's test) and results are expressed and compound potencies are expressed as the MED (minimum effective dose), in mg/Kg/day, that yields a percent (%) decrease in hyperalgesia that is statistically significant.

Table 1 shows the results from Tests A, B and C for certain compounds of the invention. Where no data is provided in the table, the test was not performed.

TABLE 1

| | | Test B | Test B | Test C |
|---|---|---|---|---|
| | Test A | First phase | Second phase | MED |
| | Ki (nM) | Dose (% Inh.) | Dose (% Inh.) | (% Inh.) |
| Ex. 1 | 79 | 70 (52%) | 70 (57%) | 30 (71%) |
| Ex. 2 | 99 | 200 (74%) | 200 (71%) | 30 (59%) |
| Ex. 3 | 146 | 200 (56%) | 200 (46%) | 30 (31%) |
| Ex. 4 | 44 | | | 30 (44%) |
| Ex. 5 | 371 | | | |
| Ex. 6 | 189 | | | 30 (64%) |
| Ex. 7 | 39 | | | 15 (49%) |
| Ex. 8 | 513 | | | |
| Ex. 9 | 61 | 200 (56%) | 200 (46%) | 15 (77%) |
| Ex. 10 | 24 | | | 30 (41%) |
| Ex. 11 | 29 | | | 15 (−17%) |
| Ex. 12 | 34 | | | 15 (17%) |
| Ex. 13 | 56 | | | 15 (1%) |
| Ex. 14 | 128 | | | 30 (26%) |
| Ex. 15 | 103 | | | |

1. A method for treating a subject suffering from pain comprising administering a pain-ameliorating effective amount of any compound according to structural diagram I; 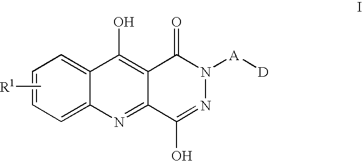
wherein:
A is (CH₂)ₙ where n has a value selected from 0, 1, 2, 3 or 4, and
D is selected from an 5- or 6-membered heteroaryl moiety or a benz-derivative thereof, having 1, 2 or 3 ring atoms selected from oxygen, nitrogen or sulfur, and
R¹ is halo.

2. A method according to claim 1, comprising administering a pain-ameliorating effective amount of a compound according to structural diagram I wherein:
D is selected from pyridyl, quinolyly, pyrazinyl, pyradizinyl, furanyl, benz[b]furanyl, imidazolyl, oxazolyl, thienyl, benz[b]thienyl and thiazolyl.

3. A method according to claim 1, comprising administering a pain-ameliorating effective amount of a compound according to structural diagram II wherein:

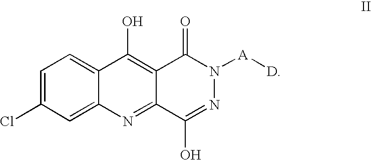

4. A method according to claim 3, comprising administering a pain-ameliorating effective amount of a compound according to structural diagram II wherein:
D is selected from pyridyl, quinolyly, pyrazinyl, pyradizinyl, furanyl, benz[b]furanyl, imidazolyl, oxazolyl, thienyl, benz[b]thienyl and thiazolyl.

5. A method according to claim 3, comprising administering a pain-ameliorating effective amount of a compound selected from:
7-Chloro-4-hydroxy-2-(4-pyridylmethyl)-1,2,5,10-tetrahydropyridazino[4,5-b]quinoline-1,10-dione;
7-Chloro-4-hydroxy-2-(3-pyridylmethyl)-1,2,5,10-tetrahydropyridazino[4,5-b]quinoline-1,10-dione;
7-Chloro-4-hydroxy-2-(2-pyridylmethyl)-1,2,5,10-tetrahydropyridazino[4,5-b]quinoline-1,10-dione;
7-Chloro-4hydroxy-2-benzo[d]furan-2-ylmethyl-1,2,5,10-tetrahydropyridazino[4,5-b]quinoline-1,10-dione;
7-Chloro-4-hydroxy-2-(quinolin-4-ylmethyl)-1,2,5,10-tetrahydropyridazino[4,5-b]quinoline-1,10-dione;
7-Chloro-4-dimethylcarbamoyl-2-pyridin-4-ylmethyl-1,2,5,10-tetrahydropyridazino[4,5-b]quinoline-1,10-dione;
7-Chloro-4-hydroxy-2-(pyrazin-2-ylmethyl)-1,2,5,10-tetrahydropyridazino[4,5-b]quinoline-1,10-dione;
7-Chloro-4-hydroxy-2-(5-isoxazolino)methyl-1,2,5,10-tetrahydropyridazino[4,5-b]quinoline-1,10-dione;
7-Chloro-4-hydroxy-2-(pyrimidin-2-ylmethyl)-1,2,5,10-tetrahydropyridazino[4,5-b]quinoline-1,10-dione;
7-Chloro-4-hydroxy-2-(furan-2-ylmethyl)-1,2,5,10-tetrahydropyridazino[4,5-b]quinoline-1,10-dione;
7-Chloro-4-hydroxy-2-(3-furylmethyl)-1,2,5,10-tetrahydropyridazino[4,5-b]quinoline-1,10-dione;
7-Chloro-4-hydroxy-2-(thien-2-ylmethyl)-1,2,5,10-tetrahydropyridazino[4,5-b]quinoline-1,10-dione;
7-Chloro-4-hydroxy-2-(thien-3-ylmethyl)-1,2,5,10-tetrahydropyridazino[4,5-b]quinoline-1,10-dione;
7-Chloro-4-hydroxy-2-(benzo[b]thien-2-ylmethyl)-1,2,5,10-tetrahydropyridazino[4,5-b]quinoline-12,10 dione;
7-Chloro-4hydroxy-2-(1,3-thiazo-2-ylmethyl)-1,2,5,10-tetrahydropyridazino[4,5-b]quinoline-1,10-dione, and
7-Chloro-4-hydroxy-2-(imidazol-2-ylmethyl)-1,2,5,10-tetrahydropyridazino[4,5-b]quinoline-1,10-dione.

6. A pharmaceutical composition comprising a pain-ameliorating effective amount of a compound according to structural diagram I together with a pharmaceutically-acceptable excipient or diluent; 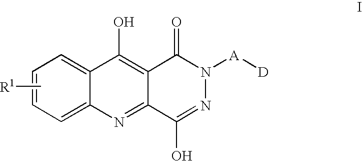
wherein:
A is (CH₂)ₙ where n has a value selected from 0, 1, 2, 3 or 4, and
D is selected from an 5- or 6-membered heteroaryl moiety or a benz-derivative thereof, having 1, 2 or 3 ring atoms selected from oxygen, nitrogen or sulfur, and
R¹ is halo.
